(19) United States
(12) Patent Application Publication
(10) Pub. No.: US 2010/0016965 A1
(43) Pub. Date: Jan. 21, 2010

(54) Accommodative IOL with Toric Optic and Extended Depth of Focus

(76) Inventors: Xin Hong, Fort Worth, TX (US); Mutlu Karakelle, Fort Worth, TX (US); Xiaoxiao Zhang, Fort Worth, TX (US); Myoung-Taek Choi, Arlington, TX (US); Yan Zhang, Vernon, CT (US); Son Tran, Arlington, TX (US)

Correspondence Address: ALCON, IP LEGAL, TB4-8, 6201 SOUTH FREEWAY, FORT WORTH, TX, 76134, US

(21) Appl. No.: 12/503,307

(22) Filed: Jul. 15, 2009

Related U.S. Application Data
(60) Provisional application No. 61/080,796, filed on Jul. 15, 2008.

(51) Int. Cl. A61F 2/16

(52) U.S. Cl. 623/6.34; 623/6.37

(57) ABSTRACT

In one aspect, the present invention provides an intraocular lens (IOL), which comprises at least two optics disposed in tandem along an optical axis, and an accommodative mechanism that is coupled to at least one of the optics and is adapted to adjust a combined optical power of the optics in response to natural accommodative forces of an eye in which the optics are implanted so as to provide accommodation. At least one of the optics has a surface characterized by a first refractive region, a second refractive region and transition region therebetween, where an optical phase shift of incident light having a design wavelength (e.g., 550 nm) across the transition region corresponds to a non-integer fraction of that wavelength.

### RELATED APPLICATION

This application is related to U.S. patent application entitled “An Extended Depth Of Focus (EDOF) Lens To Increase Pseudo-Accommodation By Utilizing Pupil Dynamics,” which is concurrently filed herewith and is herein incorporated by reference.

### BACKGROUND

The present invention relates generally to ophthalmic lenses, and more particularly, to accommodative intraocular lenses (IOLs) that provide enhanced vision via controlled variation of the phase shift across a transition region provided on at least one of the lens surfaces.

The optical power of the eye is determined by the optical power of the cornea and that of the crystalline lens, with the lens providing about a third of the eye's total optical power. The lens is a transparent, biconvex structure whose curvature can be changed by ciliary muscles for adjusting its optical power so as to allow the eye to focus on objects at varying distances.

The natural lens, however, becomes less transparent in individuals suffering from cataract, e.g., due to age and/or disease, thus diminishing the amount of light that reaches the retina. A known treatment for cataract involves removing the opacified natural lens and replacing it with an artificial intraocular lens (IOL). Many IOLs, commonly known as monofocal IOLs, provide a single optical power and hence do not allow accommodation. Multifocal IOLs are also known that provide primarily two optical powers, typically a far and a near optical power. Another class of IOLs, commonly known as accommodative IOLs, can provide a certain degree of accommodation in response to the eye's natural accommodative forces. However, the range of accommodation provided by such accommodative IOLs can be limited, e.g., due to spatial restrictions imposed by ocular anatomy.

Accordingly, there is a need for improved accommodative IOLs.

### SUMMARY

In one aspect, the present invention provides an intraocular lens (IOL), which comprises at least two optics disposed in tandem along an optical axis, and an accommodative mechanism that is coupled to at least one of the optics and is adapted to adjust a combined optical power of the optics in response to natural accommodative forces of an eye in which the optics are implanted so as to provide accommodation. At least one of the optics has a surface characterized by a first refractive region, a second refractive region and a transition region therebetween, where an optical phase shift of incident light having a design wavelength (e.g., 550 nm) across the transition region corresponds to a non-integer fraction of that wavelength. In designing IOLs and lenses generally, optical performance can be determined by measurements using a so-called “model eye” or by calculations, such as predictive ray tracing. Typically, such measurements and calculations are performed based on light from a narrow selected region of the visible spectrum to minimize chromatic aberrations. This narrow region is known as the “design wavelength.”

In the above accommodative IOL, at least one of the optics can provide a positive optical power (e.g., an optical power in a range of about +20 D to about +60 D) and at least another one of the optics can provide a negative optical power (e.g., an optical power in a range of about −26 D to about −2 D). In some cases, the accommodative mechanism is adapted to move at least one of the optics along the optical axis in response to the eye's natural accommodative forces so as to provide accommodation.

In a related aspect, in the above IOL, the surface having the transition region exhibits a profile (Z_{sag}) defined by the following relation:

Z_{sag}=Z_{base}+Zₐᵤₓ,

wherein,

Z_{sag} denotes a sag of the surface relative to the optical axis as a function of radial distance from said axis and Z_{base} denotes a base profile of the surface, and wherein,

$${Z}_{aux} = \{ \begin{matrix}0 , & 0 \leq r < {r}_{1} \\ \frac{Δ}{( {r}_{2 } - {r}_{1} )} ( r - {r}_{1} ) , & {r}_{1} \leq r < {r}_{2} \\ Δ , & {r}_{2} < r\end{matrix}$$

wherein,

r₁ denotes an inner radial boundary of the transition region,

r₂ denotes an outer radial boundary of the transition region, and wherein,

Δ is defined by the following relation:

$$Δ = \frac{α λ}{( {n}_{2} - {n}_{1} )} ,$$

wherein,

n₁ denotes an index of refraction of material forming the optic,

n₂ denotes an index of refraction of a medium surrounding the optic,

λ denotes a design wavelength, and

α denotes a non-integer fraction.

In a related aspect, the base profile (Z_{base}) of the above surface having the transition region can be defined by the following relation:

$${z}_{base} = \frac{{cr}^{2}}{1 + \sqrt{1 - ( 1 + k ) {c}^{2} {r}^{2}}} + {a}_{2} {r}^{2} + {a}_{4} {r}^{4} + {a}_{6} {r}^{6} + … ,$$

wherein,

r denotes a radial distance from the optical axis,

c denotes a base curvature of the surface,

k denotes a conic constant,

a₂ is a second order deformation constant,

a₄ is a fourth order deformation constant,

a₆ is a sixth order deformation constant.

In another embodiment, the IOL surface having the transition region has a surface profile (Z_{sag}) defined by the following relation:

Z_{sag}=Z_{base}+Zₐᵤₓ,

wherein,

Z_{sag} denotes a sag of the surface relative to the optical axis as a function of radial distance from said axis, and

wherein,

$${z}_{base} = \frac{{cr}^{2}}{1 + \sqrt{1 - ( 1 + k ) {c}^{2} {r}^{2}}} + {a}_{2} {r}^{2} + {a}_{4} {r}^{4} + {a}_{6} {r}^{6} + … ,$$

wherein,

r denotes a radial distance from the optical axis,

c denotes a base curvature of the surface,

k denotes a conic constant,

a₂ is a second order deformation constant,

a₄ is a fourth order deformation constant,

a₆ is a sixth order deformation constant, and

wherein,

$${z}_{aux} = \{ \begin{matrix}0 , & 0 \leq r < {r}_{1 a} \\ \frac{{Δ}_{1}}{( {r}_{1 b} - {r}_{1 a} )} ( r - {r}_{1 a} ) , & {r}_{1 a} \leq r < {r}_{1 b} \\ {Δ}_{1} , & {r}_{1 b} \leq r < {r}_{2 a} \\ {Δ}_{1} + \frac{( {Δ}_{2} - {Δ}_{1} )}{( {r}_{2 b} - {r}_{2 a} )} ( r - {r}_{2 a} ) & {r}_{2 a} \leq r < {r}_{2 b} \\ {Δ}_{2 } & {r}_{2 b} < r\end{matrix}$$

wherein

r denotes the radial distance from an optical axis of the lens,

r₁ₐ denotes the inner radius of a first substantially linear portion of transition region of the auxiliary profile,

r_{1b} denotes the outer radius of the first linear portion,

r₂ₐ denotes the inner radius of a second substantially linear portion of the transition region of the auxiliary profile, and

r_{2b} denotes the outer radius of the second linear portion, and

wherein

each of Δ₁ and Δ₂ can is defined in accordance with the following relation:

$${Δ}_{1 } = \frac{{α}_{1} λ}{( {n}_{2} - {n}_{1} )} , \\ {Δ}_{2} = \frac{{α}_{2} λ}{( {n}_{2} - {n}_{1} )} , and$$

wherein,

n₁ denotes an index of refraction of material forming the optic,

n₂ denotes an index of refraction of a medium surrounding the optic,

λ denotes a design wavelength (e.g., 550 nm),

α₁ denotes a non-integer fraction (e.g., ½, 3/2 . . . ), and

α₂ denotes a non-integer fraction (e.g., ½, 3/2, . . . ).

By way of example, in the above relations, the base curvature c can be in a range of about 0.0152 mm⁻¹ to about 0.0659 mm⁻¹, and the conic constant k can be in a range of about −1162 to about −19, a₂ can be in a range of about −0.00032 mm⁻¹ to about 0.0 mm⁻¹, a₄ can be in a range of about 0.0 mm⁻³ to about −0.000053 (minus 5.3×10⁻⁵) mm⁻³, and a₆ can be in a range of about 0.0 mm⁻⁵ to about 0.000153 (1.53×10⁻⁴) mm⁻⁵.

In another aspect, in the above accommodative IOLs, the accommodative mechanism can include a ring for positioning in the capsular bag, and a plurality of flexible members that couple the ring to at least one of the optics. The ring is adapted to cause the flexible members to move the optic coupled thereto in response to natural accommodative forces exerted by the capsular bag onto the ring so as to provide accommodation. In some cases, the accommodative mechanism can provide a dynamic accommodation in a range of about 0.5 D to about 2.5 D while the aforementioned transition region can extend the IOL's depth-of-focus by at least about 0.5 D (e.g., in a range of about 0.5 D to about 1.25 D), e.g., for pupil sizes in a range of about 2.5 mm to about 3.5 mm, to provide a degree of pseudoaccommodation.

In another aspect, an intraocular lens system is disclosed that includes an optical system adapted for positioning in the capsular bag of a patient's eye, where the optical system comprises a plurality of lenses. The lens system further includes an accommodative mechanism coupled to the optical system to cause a change in its optical power in response to natural accommodative forces of the eye so as to provide accommodation. The optical system has at least one toric surface and at least one surface having a first refractive region, a second refractive region and a transition region therebetween, such that an optical phase shift of incident light having a design wavelength (e.g., 550 nm) across the transition region corresponds to a non-integer fraction of that wavelength.

Further understanding of the various aspects of the invention can be obtained by reference to the following detailed description in conjunction with the associated drawings, which are described briefly below.

### BRIEF DESCRIPTION OF THE DRAWINGS

FIG. 1A is a schematic cross-sectional view of an IOL according to an embodiment of the invention,

FIG. 1B is schematic top view of the anterior surface of the IOL shown in FIG. 1A,

FIG. 2A schematically depicts phase advancement induced in a wavefront incident on a surface of a lens according to one implementation of an embodiment of the invention via a transition region provided on that surface according to the teachings of the invention,

FIG. 2B schematically depicts phase delay induced in a wavefront incident on a surface of a lens according to another implementation of an embodiment of the invention via a transition region provided on the surface according to the teachings of the invention,

FIG. 3 schematically depicts that the profile of at least a surface of a lens according to an embodiment of the invention can be characterized by superposition of a base profile and an auxiliary profile,

FIGS. 4A-4C provide calculated through-focus MTF plots for a hypothetical lens according to an embodiment of the invention for different pupil sizes,

FIGS. 5A-5F provide calculated through-focus MTF plots for hypothetical lenses according to some embodiments of the invention, where each lens has a surface characterized by a base profile and an auxiliary profile defining a transition region providing a different Optical Path Difference (OPD) between an inner and an outer region of the auxiliary profile relative to the respective OPD in the other lenses,

FIG. 6 is a schematic cross-sectional view of an IOL according to another embodiment of the invention, and

FIG. 7 schematically depicts that the profile of the anterior surface can be characterized as a superposition of a base profile and an auxiliary profile that includes a two-step transition region.

FIG. 8 presents calculated through-focus monochromatic MTF plots for a hypothetical lens according to an embodiment of the invention having a two-step transition region,

FIG. 9A is a schematic cross-sectional view of an accommodative intraocular lens (IOL) in accordance with one embodiment of the invention,

FIG. 9B is a schematic elevational view of the accommodation IOL of FIG. 10A,

FIG. 10A schematically depicts an anterior optic of the IOL of FIGS. 10A-10B coupled to the lens's accommodative mechanism,

FIG. 10B is a schematic side view of the anterior optic shown in FIG. 11A,

FIG. 10 C is a schematic top view of the anterior optic shown in FIG. 11B, and

FIG. 11 schematically presents a toric surface characterized by different radii of curvature along two orthogonal directions along the surface.

FIG. 12A is a schematic top view of an accommodative IOL according to another embodiment of the invention, and

FIG. 12B is a schematic side view of the optic employed in the accommodative IOL of FIG. 13A.

### DETAILED DESCRIPTION

The present invention is generally directed to ophthalmic lenses (such as IOLs) and methods for correcting vision that employ such lenses. In the embodiments that follow, the salient features of various aspects of the invention are discussed in connection with intraocular lenses (IOLs). The teachings of the invention can also be applied to other ophthalmic lenses, such as contact lenses. The term “intraocular lens” and its abbreviation “IOL” are used herein interchangeably to describe lenses that are implanted into the interior of the eye to either replace the eye's natural lens or to otherwise augment vision regardless of whether or not the natural lens is removed. Intracorneal lenses and phakic intraocular lenses are examples of lenses that may be implanted into the eye without removal of the natural lens. In many embodiments, the lens can include a controlled pattern of surface modulations that selectively impart an optical path difference between an inner and an outer portion of the lens's optic such that the lens would provide sharp images for small and large pupil diameters as well as pseudo-accommodation for viewing objects with intermediate pupil diameters.

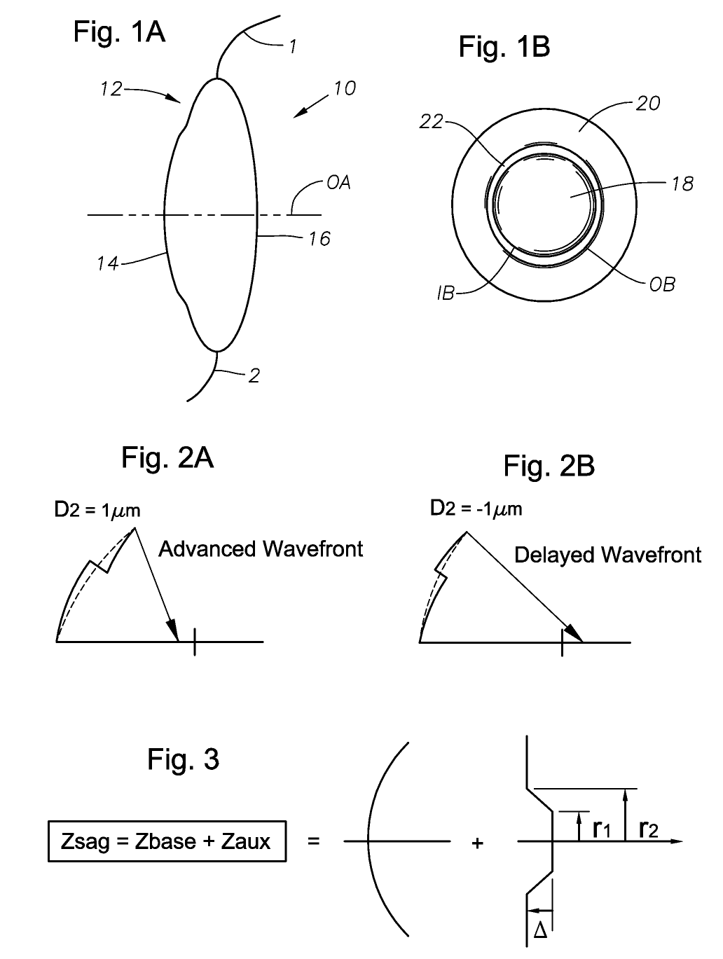

FIGS. 1A and 1B schematically depict an intraocular lens (IOL) 10 according to an embodiment of the invention that includes an optic 12 having an anterior surface 14 and a posterior surface 16 that are disposed about an optical axis OA. As shown in FIG. 1B, the anterior surface 14 includes an inner refractive region 18, an outer annular refractive region 20, and an annular transition region 22 that extends between the inner and outer refractive regions. In contrast, the posterior surface 16 is in the form of a smooth convex surface. In some embodiments, the optic 12 can have a diameter D in a range of about 1 mm to about 5 mm, though other diameters can also be utilized.

The exemplary IOL 10 also includes one or more fixation members 1 and 2 (e.g., haptics) that can facilitate its placement in the eye.

In this embodiment, each of the anterior and the posterior surfaces includes a convex base profile, though in other embodiments concave or flat base profiles can be employed. While the profile of the posterior surface is defined solely by a base profile, the profile of the anterior surface is defined by addition of an auxiliary profile to its base profile so as to generate the aforementioned inner, outer and the transition regions, as discussed further below. The base profiles of the two surfaces in combination with the index of refraction of the material forming the optic can provide the optic with a nominal optical power. The nominal optical power can be defined as the monofocal refractive power of a putative optic formed of the same material as the optic 12 with the same base profiles for the anterior and the posterior surface but without the aforementioned auxiliary profile of the anterior surface. The nominal optical power of the optic can also be viewed as the monofocal refractive power of the optic 12 for small apertures with diameters less than the diameter of the central region of the anterior surface.

The auxiliary profile of the anterior surface can adjust this nominal optical power such that the optic's actual optical power, as characterized, e.g. by a focal length corresponding to the axial location of the peak of a through-focus modulation transfer function calculated or measured for the optic at a design wavelength (e.g., 550 nm), would deviate from the lens's nominal optical power, particularly for aperture (pupil) sizes in an intermediate range, as discussed further below. In many embodiments, this shift in the optical power is designed to improve near vision for intermediate pupil sizes. In some cases, the nominal optical power of the optic can be in a range of about −15 D to about +50 D, and preferably in a range of about 6 D to about 34 D. Further, in some cases, the shift caused by the auxiliary profile of the anterior surface to the optic's nominal power can be in a range of about 0.25 D to about 2.5 D.

With continued reference to FIGS. 1A and 1B, the transition region 22 is in the form of an annular region that extends radially from an inner radial boundary (IB) (which in this case corresponds to an outer radial boundary of the inner refractive region 18) to an outer radial boundary (OB) (which in this case corresponds to inner radial boundary of the outer refractive region). While in some cases, one or both boundaries can include a discontinuity in the anterior surface profile (e.g., a step), in many embodiments the anterior surface profile is continuous at the boundaries, though a radial derivative of the profile (that is, the rate of change of the surface sag as a function of radial distance from the optical axis) can exhibit a discontinuity at each boundary. In some cases, the annular width of the transition region can be in a range of about 0.75 mm to about 2.5 mm. In some cases, the ratio of an annular width of the transition region relative to the radial diameter of the anterior surface can be in a range of about 0 to about 0.2.

In many embodiments, the transition region 22 of the anterior surface 14 can be shaped such that a phase of radiation incident thereon would vary monotonically from its inner boundary (IB) to its outer boundary (OB). That is, a non-zero phase difference between the outer region and the inner region would be achieved via a progressive increase or a progressive decrease of the phase as a function of increasing radial distance from the optical axis across the transition region. In some embodiments, the transition region can include plateau portions, interspersed between portions of progressive increase or decrease of the phase, in which the phase can remain substantially constant.

In many embodiments, the transition region is configured such that the phase shift between two parallel rays, one of which is incident on the outer boundary of the transition region and the other is incident on the inner boundary of the transition region, can be a non-integer rational fraction of a design wavelength (e.g., a design wavelength of 550 nm). By way of example, such a phase shift can be defined in accordance with the following relation:

$$\begin{matrix}Phase Shift = \frac{2 π}{λ} OPD , & Eq . ( 1 A ) \\ OPD = ( A + B ) λ & Eq . ( 1 B )\end{matrix}$$

wherein,

A designates an integer,

B designates a non-integer rational fraction, and

λ designates a design wavelength (e.g., 550 nm).

By way of example, the total phase shift across the transition region can be

$$\frac{λ}{2} , \frac{λ}{3} ,$$

etc, where λ represents a design wavelength, e.g., 550 nm. In many embodiments, the phase shift can be a periodic function of the wavelength of incident radiation, with a periodicity corresponding to one wavelength.

In many embodiments, the transition region can cause a distortion in the wavefront emerging from the optic in response to incident radiation (that is, the wavefront emerging from the posterior surface of the optic) that can result in shifting the effective focusing power of the lens relative to its nominal power. Further, the distortion of the wavefront can enhance the optic's depth of focus for aperture diameters that encompass the transition region, especially for intermediate diameter apertures, as discussed further below. For example, the transition region can cause a phase shift between the wavefront emerging from the outer portion of the optic and that emerging from its inner portion. Such a phase shift can cause the radiation emerging from optic's outer portion to interfere with the radiation emerging from the optic's inner portion at the location at which the radiation emerging from the optic's inner portion would focus, thus resulting in an enhanced depth-of-focus, e.g., as characterized by an asymmetric MTF (modulation transfer function) profile referenced to the peak MTF. The term “depth-of-focus” and “depth-of-field” can be used interchangeably and are known and readily understood by those skilled in the art as referring to the distances in the object and image spaces over which an acceptable image can be resolved. To the extent that any further explanation may be needed, the depth-of-focus can refer to an amount of defocus relative to a peak of a through-focus modulation transfer function (MTF) of the lens measured with a 3 mm aperture and green light, e.g., light having a wavelength of about 550 nm, at which the MTF exhibits a contrast level of at least about 15% at a spatial frequency of about 50 lp/mm. Other definitions can also be applied and it should be clear that depth of field can be influenced by many factors including, for example, aperture size, chromatic content of the light forming the image, and base power of the lens itself.

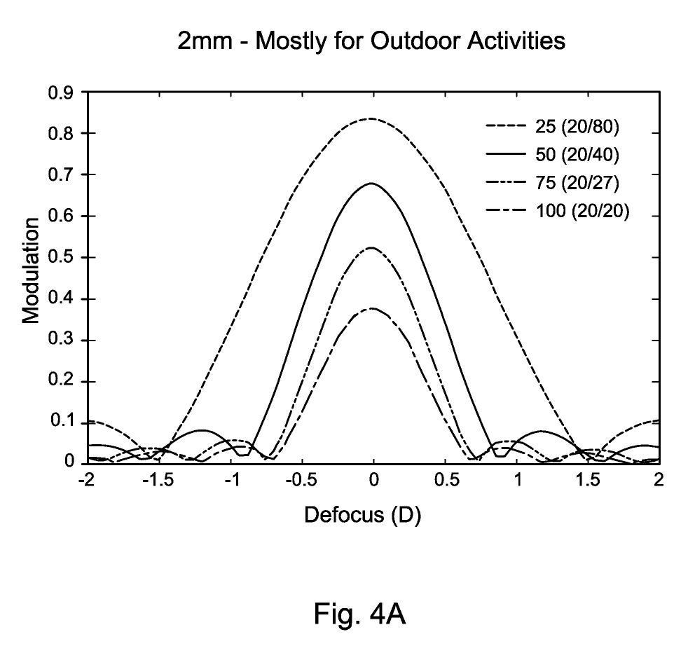

By way of further illustration, FIG. 2A schematically shows a fragment of a wavefront generated by an anterior surface of an IOL according to an embodiment of the invention having a transition region between an inner portion and an outer portion of the surface, and a fragment of a wavefront incident on that surface, and a reference spherical wavefront (depicted by dashed lines) that minimizes the RMS (root-mean-square) error of the actual wavefront. The transition region gives rise to a phase advancement of the wavefront (relative to that corresponding to a putative similar surface without the transition region) that leads to the convergence of the wavefront at a focal plane in front of the retinal plane (in front of the nominal focal plane of the IOL in absence of the transition region). FIG. 2B schematically shows another case in which the transition region gives rise to a phase delay of an incident wavefront that leads to the convergence of the wavefront at a focal plane beyond the retinal plane (beyond the nominal focal plane of the IOL in absence of the transition region).

By way of illustration, in this implementation, the base profile of the anterior and/or the posterior surfaces can be defined by the following relation:

$${z}_{base} = \frac{{cr}^{2}}{1 + \sqrt{1 - ( 1 + k ) {c}^{2} {r}^{2}}} + f ( {r}^{2} , {r}^{4} , {r}^{6} , … ) \qquad Eq . ( 2 )$$

wherein,

c denotes the curvature of the profile,

k denotes the conic constant, and

wherein,

ƒ(r², r⁴, r⁶, . . . ) denotes a function containing higher order contributions to the base profile. By way of example, the function f can be defined by the following relation:

ƒ(r²,r⁴,r⁶, . . . )=a₂r²+a₄r⁴+a₆r⁶+ . . . Eq. (3)

wherein,

a₂ is a second order deformation constant,

a₄ is a fourth order deformation constant, and

a₆ is a sixth order deformation constant. Additional higher order terms can also be included.

By way of example, in some embodiments, the parameter c can be in a range of about 0.0152 mm⁻¹ to about 0.0659 mm⁻¹, the parameter k can be in range of about −1162 to about −19, a₂ can be in a range of about −0.00032 mm⁻¹ to about 0.0 mm⁻¹, a₄ can be in a range of about 0.0 mm⁻³ to about −0.000053 (minus 5.3×10⁻⁵) mm⁻³, and a₆ can be in a range of about 0.0 mm⁻⁵ to about 0.000153 (1.53×10⁻⁴) mm⁻⁵.

The use of certain degree of asphericity in the anterior and/or posterior base profile as characterized, e.g., by the conic constant k, can ameliorate spherical aberration effects for large aperture sizes. For large aperture sizes, such asphericity can somewhat degree counteract the optical effects of the transition region, thus leading to a shaper MTF. In some other embodiments, the base profile of one or both surfaces can be toric (that is, it can exhibit different radii of curvatures along two orthogonal directions along the surface) to ameliorate astigmatic aberrations.

As noted above, in this exemplary embodiment, the profile of the anterior surface 14 can be defined by superposition of a base profile, such as the profile defined by the above Equation (1), and an auxiliary profile. In this implementation, the auxiliary profile (Zₐᵤₓ) can be defined by the following relation:

$${Z}_{aux} = \{ \begin{matrix}0 , & 0 \leq r < {r}_{1} \\ \frac{Δ }{( {r}_{2} - {r}_{1} )} ( r - {r}_{1} ) , & {r}_{1} \leq r < {r}_{2} \\ Δ , & {r}_{2} < r\end{matrix} \qquad Eq . ( 4 )$$

wherein,

r₁ denotes an inner radial boundary of the transition region,

r₂ denotes an outer radial boundary of the transition region, and wherein,

Δ is defined by the following relation:

$$Δ = \frac{α λ}{( {n}_{2} - {n}_{1} )} , \qquad Eq . ( 5 )$$

wherein,

n₁ denotes an index of refraction of material forming the optic,

n₂ denotes an index of refraction of a medium surrounding the optic,

λ denotes a design wavelength, and

α denotes a non-integer fraction, e.g., ½.

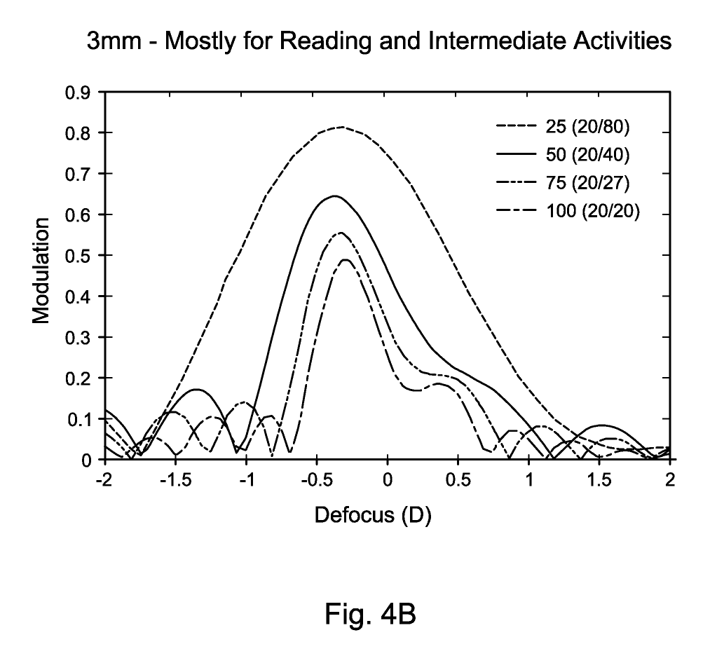

In other words, in this embodiment, the profile of the anterior surface (Z_{sag}) is defined by a superposition of the base profile (Z_{base}) and the auxiliary profile (Zₐᵤₓ) as defined below, and shown schematically in FIG. 3:

Z_{sag}=Z_{base}+Zₐᵤₓ Eq. (6)

In this embodiment, the auxiliary profile defined by the above relations (4) and (5) is characterized by a substantially linear phase shift across the transition region. More specifically, the auxiliary profile provides a phase shift that increases linearly from the inner boundary of the transition region to its outer boundary with the optical path difference between the inner and the outer boundaries corresponding to a non-integer fraction of the design wavelength.

In many embodiments, a lens according to the teachings of the invention, such the above lens 10, can provide good far vision performance by effectively functioning as a monofocal lens without the optical effects caused by the phase shift for small pupil diameters that fall within the diameter of the lens's central region (e.g., for a pupil diameter of 2 mm). For medium pupil diameters (e.g., for pupil diameters in a range of about 2 mm to about 4 mm (e.g., a pupil diameter of about 3 mm)), the optical effects caused by the phase shift (e.g., changes in the wavefront exiting the lens) can lead to enhanced functional near and intermediate vision. For large pupil diameters (e.g., for pupil diameters in a range of about 4 mm to about 5 mm), the lens can again provide good far vision performance as the phase shift would only account for a small fraction of the anterior surface portion that is exposed to incident light.

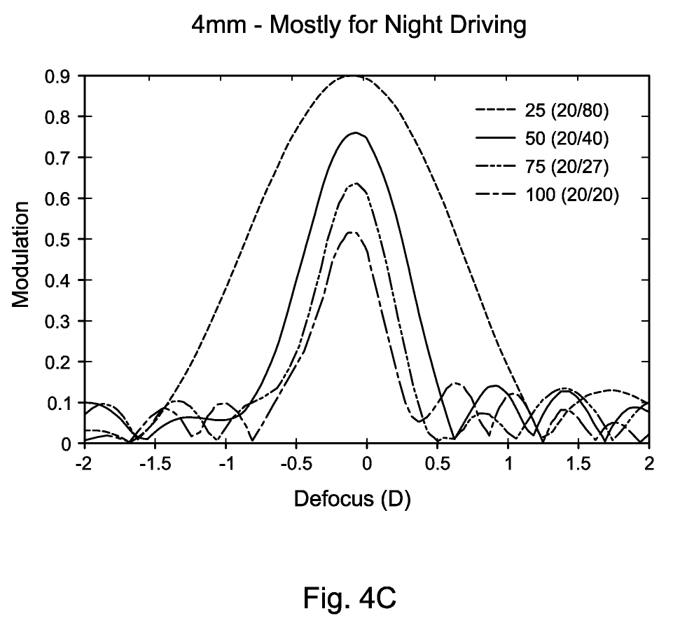

By way of illustration, FIG. 4A-4C show optical performance of a hypothetical lens according to an embodiment of the invention for different pupil sizes. The lens was assumed to have an anterior surface defined by the above relation (6), and a posterior surface characterized by a smooth convex base profile (e.g., one defined by that above relation (2)). Further, the lens was assumed to have a diameter of 6 mm with the transition region extending between an inner boundary having a diameter of about 2.2 mm to an outer boundary having a diameter of about 2.6 mm. The base curvatures of the anterior and the posterior surface were selected such that the optic would provide a nominal optical power of 21 D. Further, the medium surrounding the lens was assumed to have an index of refraction of about 1.336. Tables 1A-1C below list the various parameters of the lens's optic as well as those of its anterior and posterior surfaces:

TABLE 1A
Optic

| Central | Diameter | Index of |
|---|---|---|
| Thickness (mm) | (mm) | Refraction |
| 0.64 | 6 | 1.5418 |

TABLE 1B
Anterior Surface

| Base Profile | | | | | | | |
|---|---|---|---|---|---|---|---|
| Base | Conic | | | | | | |
| Radius | Constant | | | | Auxiliary Profile | | |
| (mm) | (k) | a₂ | a₄ | a₆ | r1 | r2 | Δ |
| 18.93 | −43.56 | 0 | 2.97E−4 | −2.3E−5 | 1.1 | 1.25 | −1.18 |

TABLE 1C
Posterior Surface

| Base | Conic | | | |
|---|---|---|---|---|
| Radius (mm) | Constant (k) | a₂ | a₄ | a₆ |
| −20.23 | 0 | 0 | 0 | 0 |

More specifically, in each of the FIGS. 4A-4C, through-focus modulation transfer (MTF) plots corresponding to the following modulation frequencies are provided: 25 lp/mm, 50 lp/mm, 75 lp/mm, and 100 lp/mm. The MTF shown in FIG. 4A for a pupil diameter of about 2 mm indicates that the lens provides good optical performance, e.g., for outdoor activities, with a depth-of focus of about 0.7 D, which is symmetric about the focal plane. For a pupil diameter of 3 mm, each of the MTFs shown in FIG. 4B is asymmetric relative to the lens's focal plane (i.e., relative to zero defocus) with a shift in its peak in the negative defocus direction. Such a shift can provide a degree of pseudoaccommodation to facilitate near vision (e.g., for reading). Further, these MTFs have greater widths than those shown by the MTFs calculated for a 2-mm pupil diameter, which translates to better performance for intermediate vision. For a larger pupil diameter of 4 mm (FIG. 4C), the asymmetry and the widths of the MTFs diminish relative to those calculated for a 3-mm diameter. This in turn indicates good far vision performance under low light conditions, e.g., for night driving.

The optical effect of the phase shift can be modulated by varying various parameters associated with that region, such as, its radial extent and the rate at which it imparts phase shift to incident light. By way of example, the transition region defined by the above relation (3) exhibits a slope defined by

$$\frac{Δ}{( {r}_{2} - {r}_{1} )} ,$$

which can be varied so as to adjust the performance of an optic having such a transition region on a surface thereof, particularly for intermediate pupil sizes.

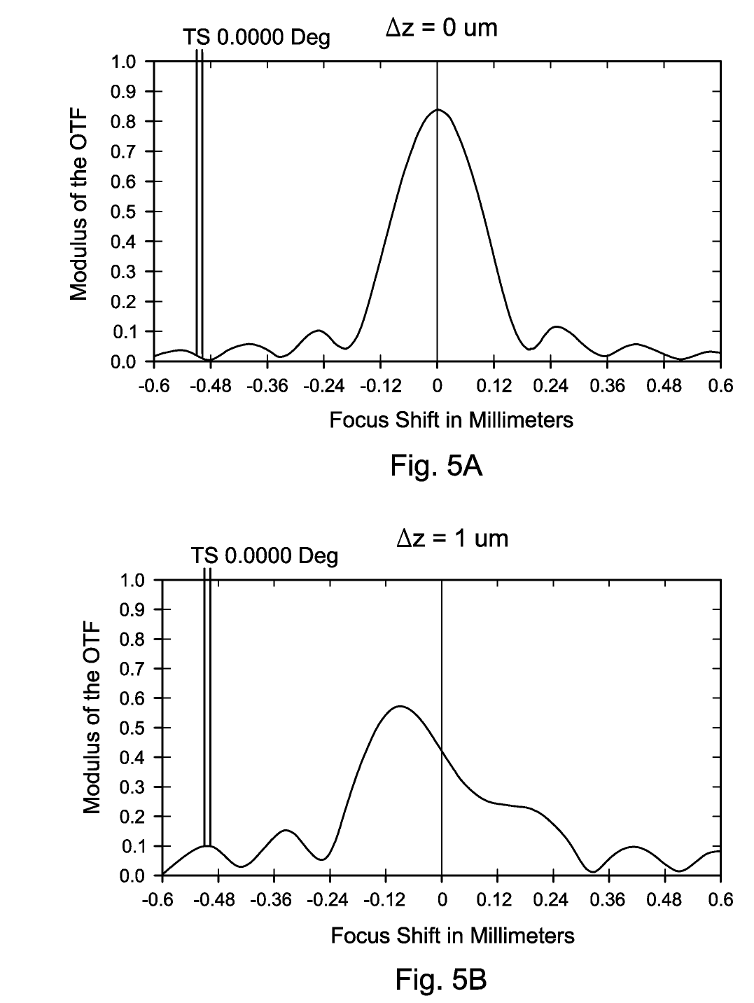

By way of illustration, FIGS. 5A-5F show calculated through-focus modulation transfer function (MTF) at a pupil size of 3 mm and for a modulation frequency of 50 lp/mm for hypothetical lenses having an anterior surface exhibiting the surface profile shown in FIG. 3 as a superposition of a base profile defined by the relation (2) and an auxiliary profile defined by the relations (4) and (5). The optic was assumed to be formed of a material having an index of refraction of 1.554. Further, the base curvature of the anterior surface and that of the posterior surface were selected such that the optic would have a nominal optical power of about 21 D.

By way of providing a reference from which the optical effects of the transition region can be more readily understood, FIG. 5A shows an MTF for an optic having a vanishing Δz, that is, an optic that lacks a phase shift according to the teachings of the invention. Such a conventional optic having smooth anterior and posterior surfaces exhibits an MTF curve that is symmetrically disposed about the optic's focal plane and exhibits a depth of focus of about 0.4 D. In contrast, FIG. 5B shows an MTF for an optic according to an embodiment of the invention in which the anterior surface includes a transition region characterized by a radial extent of about 0.01 mm and Δz=1 micron. The MTF plot shown in FIG. 5B exhibits a greater depth of focus of about 1 D, indicating that the optic provides an enhanced depth of field. Further, it is asymmetric relative to the optic's focal plane. In fact, the peak of this MTF plot is closer to the optic than its focal plane. This provides an effective optical power increase to facilitate near reading.

As the transition region becomes steeper (its radial extent remains fixed at 0.01 mm) so as to provide a ΔZ=1.5 microns (FIG. 5C), the MTF broadens further (that is, the optic provides a greater depth-of-field) and its peak shifts farther away from the optic than the optic's focal plane. As shown in FIG. 5D, the MTF for an optic having a transition region characterized by a ΔZ=2.5 microns is identical to the one shown in FIG. 5A for an optic having a ΔZ=0.

In fact, the MTF pattern is repeated for every design wavelength. By way of example, in an embodiment in which the design wavelength is 550 nm and the optic is formed of Acrysof material (cross-linked copolymer of 2-phenylethyl acrylate and 2-phenylethyl methacrylate) ΔZ=2.5 microns. For example, the MTF curve shown in FIG. 5E corresponding to a ΔZ=3.5 microns is identical to that shown in FIG. 5B for a ΔZ=1.5, and the MTF curve shown in FIG. 5F corresponding to a ΔZ=4 microns is identical to the MTF curve shown in FIG. 5C corresponding to a ΔZ=1.5 microns. The optical path difference (OPD) corresponding to ΔZ for Zₐᵤₓ defined by the above relation (3) can be defined by the following relation:

Optical Path Difference(OPD)=(n₂−n₁)ΔZ Eq. (7)

wherein

n₁ represent the index of refraction of the material from which the optic is formed, and

n₂ represents the index of refraction of the material surrounding the optic. Thus, for n₂=1.552, and n₁=1.336, and a ΔZ of 2.5 microns, an OPD corresponding to 1λ is achieved for a design wavelength of about 550 nm. In other words, the exemplary MTF plots shown in FIGS. 5A-5F are repeated for a ΔZ variation corresponding to 1λ OPD.

A transition region according to the teachings of the invention can be implemented in a variety of ways, and is not restricted to the above exemplary region that is defined by the relation (4). Further, while in some cases the transition region comprises a smoothly varying surface portion, in other cases it can be formed by a plurality of surface segments separated from one another by one or more steps.

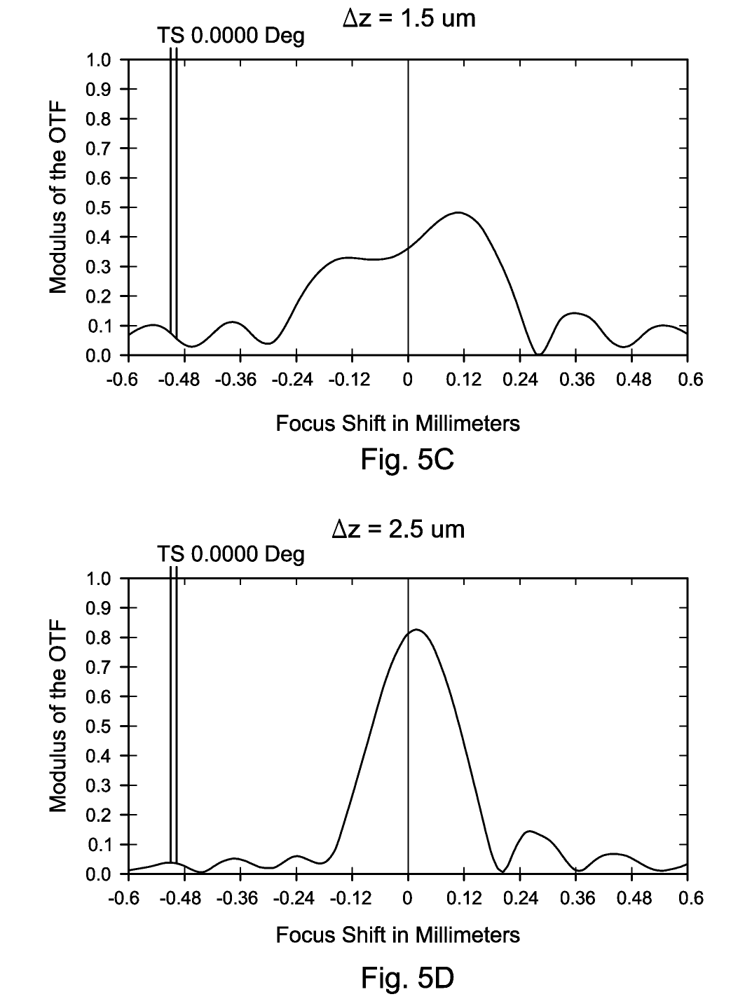

FIG. 6 schematically depicts an IOL 24 according to another embodiment of the invention that includes an optic 26 having an anterior surface 28 and a posterior surface 30. Similar to the previous embodiment, the profile of the anterior surface can be characterized as the superposition of a base profile and an auxiliary profile, albeit one that is different from the auxiliary profile described above in connection with the previous embodiment.

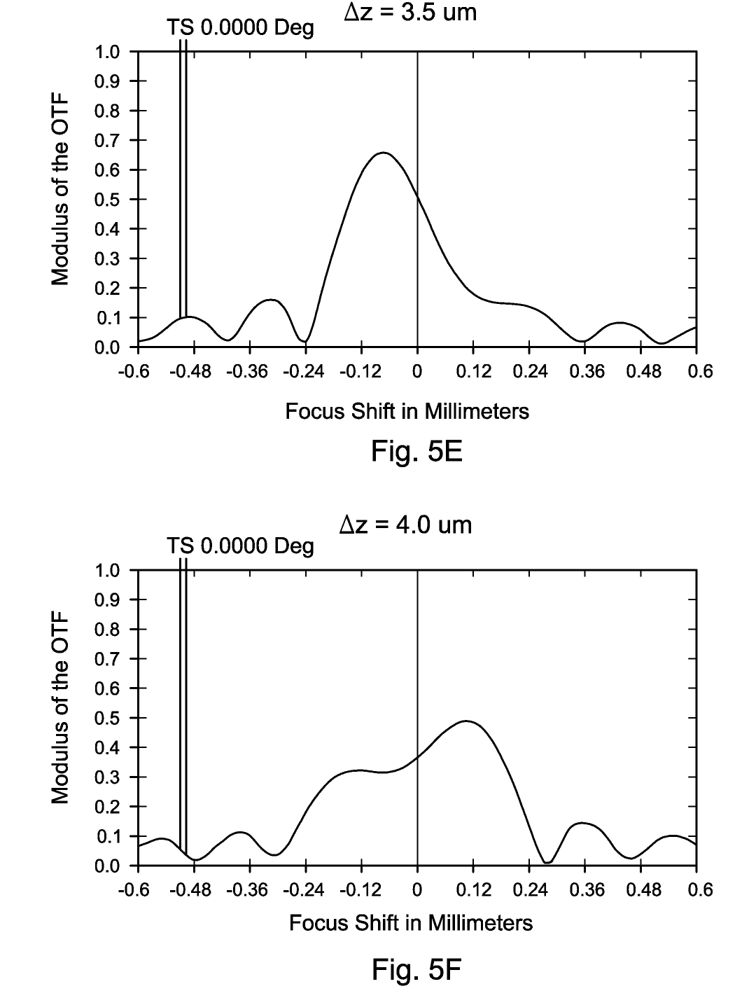

As shown schematically in FIG. 7, the profile (Z_{sag}) of the anterior surface 28 of the above IOL 24 is formed by superposition of a base profile (Z_{base}) and an auxiliary profile (Zₐᵤₓ). More specifically, in this implementation, the profile of the anterior surface 28 can be defined by the above relation (6), which is reproduced below:

Z_{sag}=Z_{base}+Zₐᵤₓ

wherein the base profile (Z_{base}) can be defined in accordance with the above relation (2). The auxiliary profile (Zₐᵤₓ) is, however, defined by the following relation:

$${z}_{aux} = \{ \begin{matrix}0 , & 0 \leq r < {r}_{1 a} \\ \frac{{Δ}_{1}}{( {r}_{1 b} - {r}_{1 a} )} ( r - {r}_{1 a} ) & {r}_{1 a} \leq r < {r}_{1 b} \\ {Δ}_{1} , & {r}_{1 b} \leq r < {r}_{2 a} \\ {Δ}_{1} + \frac{( {Δ}_{2} - {Δ}_{1} )}{( {r}_{2 b} - {r}_{2 a} )} ( r - {r}_{2 a} ) & {r}_{2 a} \leq r < {r}_{2 b} \\ {Δ}_{2} & {r}_{2 b} < r\end{matrix} \qquad Eq . ( 8 )$$

wherein r denotes the radial distance from an optical axis of the lens, and parameters r₁ₐ, r_{1b}, r₂ₐ and r_{2b} are depicted in FIG. 7, and are defined as follows:

r₁ₐ denotes the inner radius of a first substantially linear portion of the transition region of the auxiliary profile,

r_{1b} denotes the outer radius of the first linear portion,

r₂ₐ denotes the inner radius of a second substantially linear portion of the transition region of the auxiliary profile, and

r_{2b} denotes the outer radius of the second linear portion, and wherein each of Δ₁ and Δ₂ can be defined in accordance with the above relation (8).

With continued reference to FIG. 7, in this embodiment, the auxiliary profile Zₐᵤₓ includes flat central and outer regions 32 and 34 and a two-step transition 36 that connects the central and the outer regions. More specifically, the transition region 36 includes a linearly varying portion 36a, which extends from an outer radial boundary of the central region 32 to a plateau region 36b (it extends from a radial location r₁ₐ to another radial location r_{1b}). The plateau region 36b in turn extends from the radial location r_{1b} to a radial location r₂ₐ at which it connects to another linearly varying portion 36c, which extends radially outwardly to the outer region 34 at a radial location r_{2b}. The linearly varying portions 36a and 36c of the transition region can have similar or different slopes. In many implementations, the total phase shift provided across the two transition regions is a non-integer fraction of a design wavelength (e.g., 550 nm).

The profile of the posterior surface 30 can be defined by the above relation (2) for Z_{base} with appropriate choices of the various parameters, including the radius of curvature c. The radius curvature of the base profile of the anterior surface together with the curvature of the posterior surface, as well as the index of refraction of the material forming the lens, provides the lens with a nominal refractive optical power, e.g., an optical power in a range of about −15 D to about +50 D, or in a range of about 6 D to about 34 D, or in a rang of about 16 D to about 25 D.

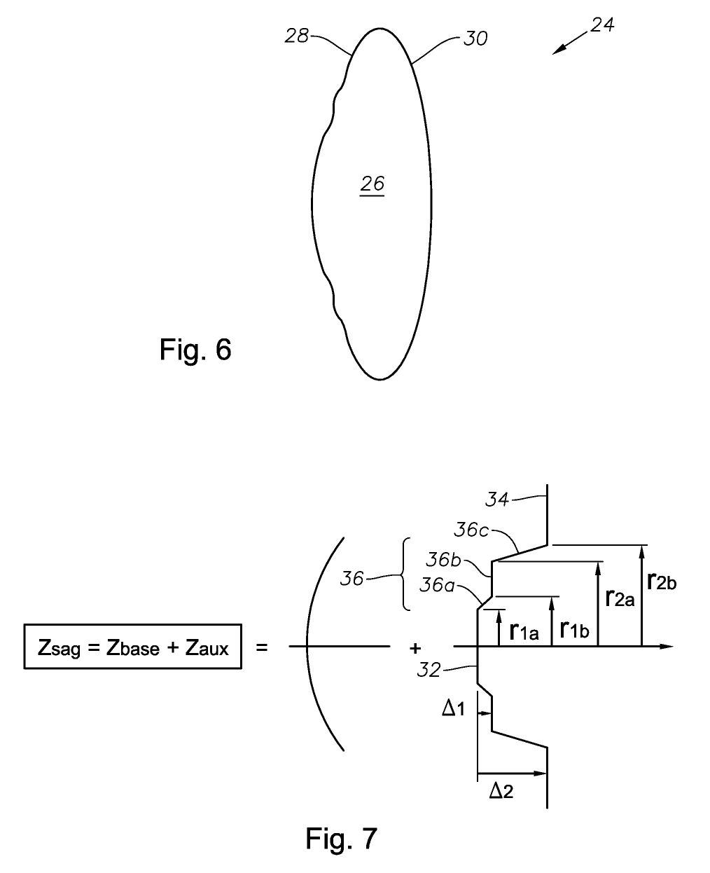

The exemplary IOL 24 can provide a number of advantages. For example, it can provide sharp far vision for small pupil sizes with the optical effects of the two-step transition region contributing to the enhancement of functional near and intermediate vision. Further, in many implementations, the IOL provides good far vision performance for large pupil sizes. By way of illustration, FIG. 8 shows through-focus MTF plots at different pupil sizes calculated for a hypothetical optic according to an embodiment of the invention having an anterior surface whose profile is defined by the above relation (2) with the auxiliary profile of the anterior surface defined by the above relation (8) and a smooth convex posterior surface. The MTF plots are computed for monochromatic incident radiation having a wavelength of 550 nm. Tables 2A-2C below provide the parameters of the anterior and the posterior surfaces of the optic:

TABLE 2A
Optic

| Central | Diameter | Index of |
|---|---|---|
| Thickness (mm) | (mm) | Refraction |
| 0.64 | 6 | 1.5418 |

TABLE 2B
Anterior Surface

| Base Profile | | | | | Auxiliary Profile | | | | | |
|---|---|---|---|---|---|---|---|---|---|---|
| Base Radius | Conic | | | | r₁ₐ | r_{1b} | r₂ₐ | r_{2b} | Δ₁ | Δ₂ |
| (mm) | Constant | a₂ | a₄ | a₆ | (mm) | (mm) | (mm) | (mm) | (micron) | (micron) |
| 18.93 | −43.564 | 0 | 2.97E−4 | −2.3E−5 | 1.0 | 1.01 | 1.25 | 1.26 | 0.67 | 2.67 |

TABLE 2C
Posterior Surface

| Base | Conic | | | |
|---|---|---|---|---|
| Radius (mm) | Constant (k) | a₂ | a₄ | a₆ |
| −20.23 | 0 | 0 | 0 | 0 |

The MTF plots show that for a pupil diameter of about 2 mm, which is equal to the diameter of the central portion of the anterior surface, the optic provides a monofocal refractive power and exhibits a relatively small depth of focus (defined as full width at half maximum) of about 0.5 D. In other words, it provides good far vision performance. As the pupil size increases to about 3 mm, the optical effects of the transition region become evident in the through-focus MTF. In particular, the 3-mm MTF is significantly broader than the 2-mm MTF, indicating an enhancement in the depth-of-field.

With continued reference to FIG. 8, as the pupil diameter increases even further to about 4 mm the incident light rays encounter not only the central and the transition regions but also part of the outer region of the anterior surface.

A variety of techniques and materials can be employed to fabricate the IOLs of the invention. For example, the optic of an IOL of the invention can be formed of a variety of biocompatible polymeric materials. Some suitable biocompatible materials include, without limitation, soft acrylic polymers, hydrogel, polymethymethacrylate, polysulfone, polystyrene, cellulose, acetate butyrate, or other biocompatible materials. By way of example, in one embodiment, the optic is formed of a soft acrylic polymer (cross-linked copolymer of 2-phenylethyl acrylate and 2-phenylethyl methacrylate) commonly known as Acrysof. The fixation members (haptics) of the IOLs can also be formed of suitable biocompatible materials, such as those discussed above. While in some cases, the optic and the fixation members of an IOL can be fabricated as an integral unit, in other cases they can be formed separately and joined together utilizing techniques known in the art.

A variety of fabrication techniques known in the art, such as a casting, can be utilized for fabricating the IOLs. In some cases, the fabrication techniques disclosed in pending patent application entitled “Lens Surface With Combined Diffractive, Toric and Aspheric Components,” filed on Dec. 21, 2007 and having a Ser. No. 11/963,098 can be employed to impart desired profiles to the anterior and posterior surfaces of the IOL.

In other aspects, the invention provides accommodative intraocular lenses and lens systems that employ an accommodative mechanism to provide dynamic accommodation in response to natural accommodative forces of the eye and include at least one optical surface according to the above teachings having a transition region that can provide a degree of pseudoaccommodation. Further, in some cases, at least one surface of such an accommodative lens (or lens system) can exhibit a toric profile for ameliorating, and preferably correcting, astigmatic aberrations. The term “dynamic accommodation” is used herein to refer to accommodation provided by a lens or lens system implanted in a patient's eye via displacement and/or deformation of at least one lens, and the term “pseudoaccommodation” is used to refer to an effective accommodation provided by at least one lens via depth of focus and/or a shift in effective optical power as a function of pupil size exhibited by that lens (e.g., an extended depth-of-focus resulting from optical profile of one or more surfaces of that lens).

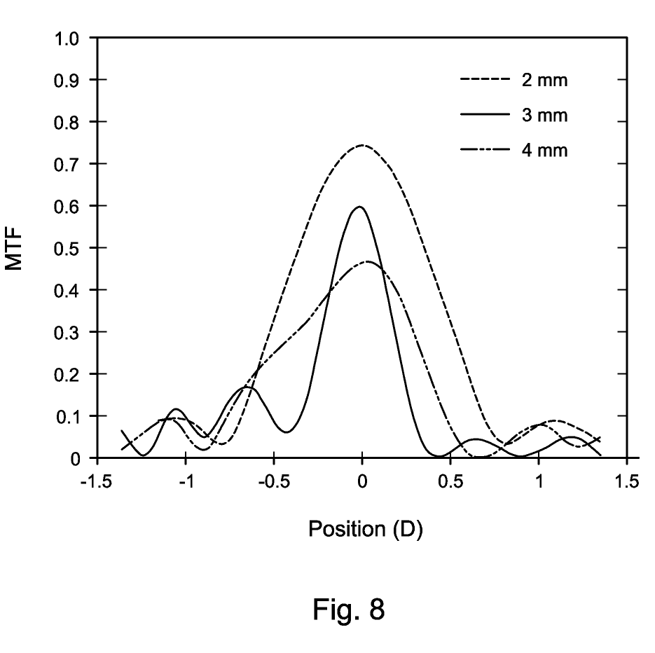

By way of example, FIGS. 9A and 9B schematically depict an exemplary dual-optic accommodative IOL 38 according to an embodiment of the invention that includes an anterior optic 40 and a posterior optic 42 disposed in tandem along an optical axis OA. In this embodiment, the anterior optic 40 provides a positive optical power while the posterior optic provides a negative optical power. As discussed further below, when the IOL is implanted in a patient's eye, the axial distance between the two optics (the distance along the optical axis OA) can vary in response to the natural accommodative forces of the eye so as to change the combined power of the optics for providing accommodation.

In some cases, the base curvatures of surfaces of the two optics together with the index of refraction of the material forming the optics are selected such that the anterior optic would provide a nominal optical power in a range of about +20 D to about +60 D and the posterior optic would provide an optical power in a range of about −26 D to about −2 D. By way of example, the optical power of each optic can be selected such that the combined nominal power of the IOL for viewing distant objects (e.g., objects at a distance greater than about 200 cm from the eye) lies in a range of about 6 D to about 34 D. This far-vision power can be achieved at the minimum axial separation of the two optics. As the axial distance between the optics increases due to the eye's natural accommodative forces, the optical power of the IOL 38 increases for viewing objects at closer distances until a maximum optical power change of the IOL is achieved. In some cases, this maximum optical power change, which corresponds to a maximum axial separation of the two optics, can be in a range of about 0.5 D to about 2.5 D.

In this embodiment, the IOL 38 can include an accommodative mechanism 44 comprising a flexible ring 46 and plurality of radially extending flexible members 48. While the posterior optics 42 is fixedly coupled to the ring, the anterior optic is coupled to the ring via the flexible members 48 that allow its axial movement relative to the posterior optic for providing accommodation, as discussed further below.

The anterior and posterior optics as well as the accommodative mechanism can be formed of any suitable biocompatible material. Some examples of such materials include, without limitation, hydrogel, silicone, polymethylmethacrylate (PMMA), and a polymeric material known as Acrysof (a cross-linked copolymer of 2-phenylethyl acrylate and 2-phenylethyl methacrylate). In some cases, the optics and the accommodative mechanism are formed of the same material while in other cases they can be formed of different materials. Further, a variety of techniques known in the art can be employed to fabricate the accommodative IOL.

In use, the IOL system 38 can be implanted in a patient's capsular bag, through a small incision made in the cornea, such that the ring would engage with the capsular bag. The ring transfers the radial accommodative forces exerted by the capsular bag thereon to the flexible members, which in turn cause the anterior optic to move axially relative to the posterior optic, thereby adjusting the IOL's optical power.

More specifically, for viewing a distant object (e.g., when the eye is in dis-accommodative state to view objects at a distance greater than about 200 cm from the eye), the eye's ciliary muscles relax to enlarge the ciliary ring diameter. The enlargement of the ciliary ring in turn causes an outward movement of the zonules, thereby flattening the capsular bag. The flattening of the capsular bag exerts a tensile force on the flexible members to move the anterior optic closer to the posterior optic, thereby lowering the optical power of the IOL. In contrast, to view closer objects (that is, when the eye is in an accommodative state), the ciliary muscles contract causing a reduction in the ciliary ring diameter. This reduction in diameter relaxes the outward radial forces on the zonules to undo the flattening of the capsular bag. This can in turn cause the accommodative mechanism to move the anterior optic away from the posterior optic, thus resulting in an increase in the optical power of the IOL system.

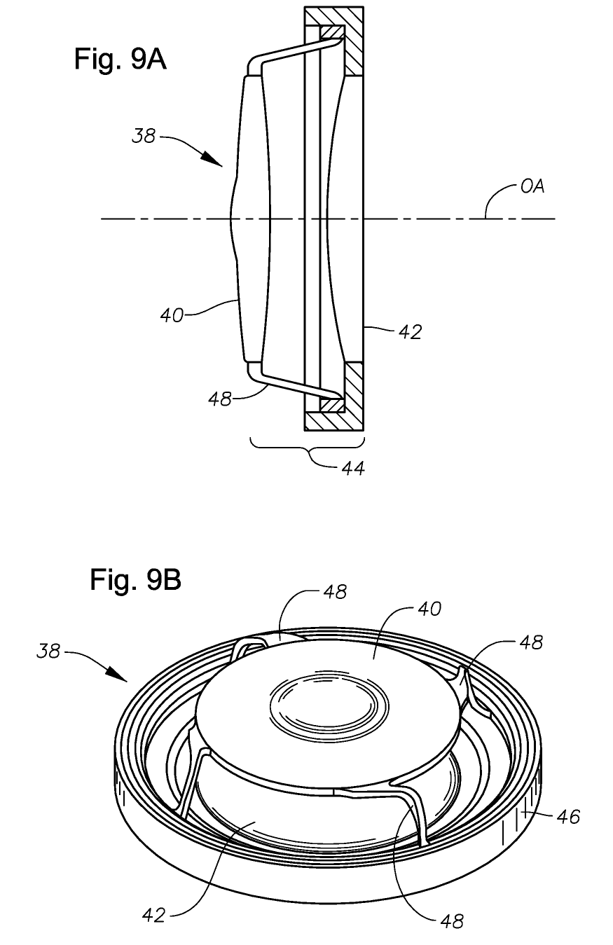

With reference to FIGS. 10A, 10B and 10C, the anterior optic 40 includes an anterior surface 40a and a posterior surface 40b. The anterior surface 40a includes a first refractive region (herein also referred to as an inner refractive region) IR, a second refractive region (herein also referred to an outer refractive region) OR and a transition region TR therebetween. As discussed further below, similar to the non-accommodative embodiments discussed above, the transition region is configured to provide a discrete phase shift for a design wavelength (e.g., 550 nm) so as to extend the depth-of-field of the anterior optic (and consequently that of the IOL 38) and shift its optical power for certain pupil sizes. This extension of the depth of field can provide a degree of pseudoaccommodation that can augment the dynamic accommodation provided by the accommodative mechanism 44.

By way of example, in this embodiment, the anterior surface 40a of the anterior optic 40 exhibits a profile (Z_{sag}) characterized by superposition of a base profile (Z_{base}) and an auxiliary profile (Zₐᵤₓ): Z_{sag}=Z_{base}+Zₐᵤₓ.

In some embodiments, the base profile can be defined in accordance with the above relations (2) and (3) with the values of various parameters within the aforementioned ranges.

Further, in some cases, the auxiliary profile can in turn be defined by the above relations (4) and (5) to include an inner and an outer refractive region that are connected via a substantially linearly varying transition region. Alternatively, the auxiliary profile can be defined by the above relation (8) to include a transition region characterized by two linearly varying portions between which a plateau region extends. It should be understood that the auxiliary profile can take other shapes so long as a phase shift imparted to incident light across its transition region would provide the requisite phase shift, e.g., a phase shift corresponding to a non-integer fraction of a design wavelength (e.g., 550 nm).

The optical effects associated with the profile of the anterior surface (e.g., a change in wavefront of incident light caused by the transition region of the auxiliary profile) can result in an extended depth-of-focus, as discussed above in detail. Such an extended depth-of-focus can provide a degree of pseudoaccommodation that can supplement the dynamic accommodation provided by the accommodative mechanism 44 to enhance the IOL's accommodative capability. By way of example, the accommodative mechanism 44 can provide a dynamic accommodation in a range of about 0.5 D to about 2.5 D while the pseudoaccommodation provided by the profile of the anterior surface can be in a range of about +0.5 D to about +1.5 D. For instance, in some cases in which the accommodative IOL 38 is implanted in a pseudophakic eye, the IOL can exhibit a dynamic accommodation of about 0.75 D and a pseudoaccommodation of about 0.75 D. The combination of the dynamic accommodation and pseudoaccommodation together with defocus exhibited by the natural eye itself (e.g., 1 D defocus for 20/40 vision) can result in, e.g., vision at 2.5 D (0.75 D+0.75 D+1 D) or 40 cm object distance. Such vision can ensure successful undertaking of most daily visual tasks.

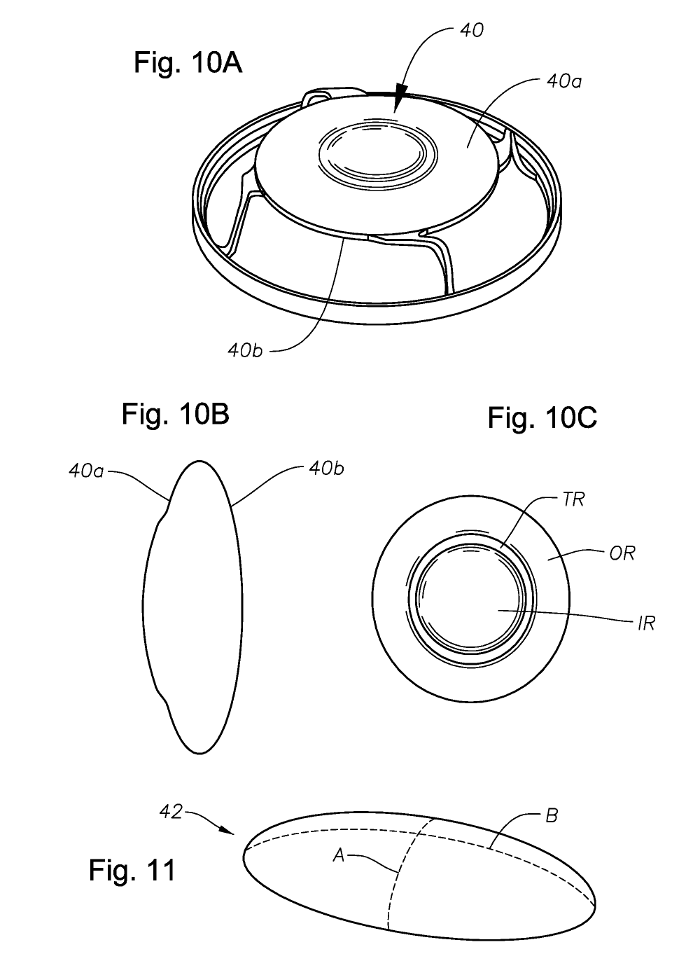

Referring again to FIGS. 10A-10C, in some embodiments, the posterior surface 40b of the anterior lens 40 exhibits a toric profile. As shown schematically in FIG. 11, such a profile of a toric surface 42 can be characterized by different radii of curvature corresponding to two orthogonal directions (e.g., directions A and B) along the surface. The toric profile can ameliorate, and preferably eliminate, astigmatic aberrations of the eye in which the IOL has been implanted. In some cases, the toricity associated with the posterior surface can be in an associated cylindrical power range of about 0.75 D to about 6 D.

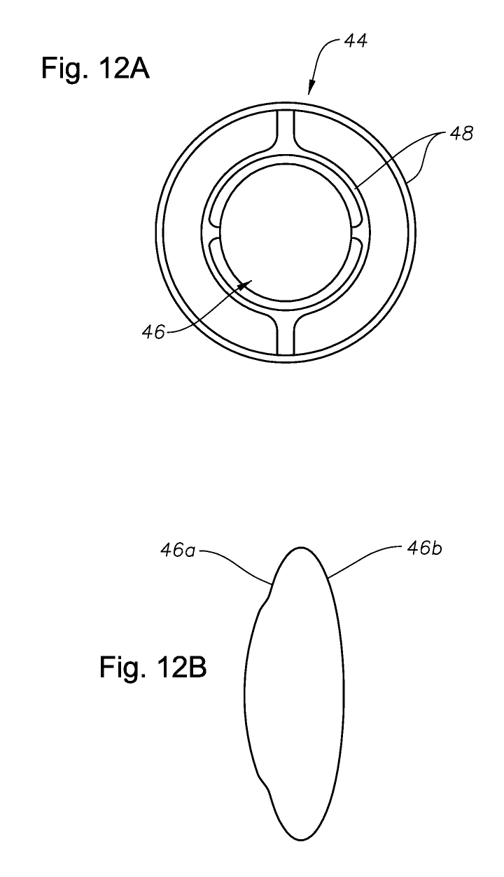

Some embodiments include, rather than a dual-optic accommodative IOL such as the above IOL 38, a single optic accommodative IOL in which a surface of the optic includes a transition region for imparting a discrete phase shift to incident light so as to extend the IOL's depth of focus and supplement the dynamic accommodation. In addition, in some cases, the other surface of that optic can exhibit a toric profile. By way of example, FIGS. 12A and 12B schematically depict an exemplary accommodative IOL 44 according to such an embodiment that includes an optic 46, which has an anterior surface 46a and a posterior surface 46b, and an accommodative mechanism 48 coupled to the optic, which can cause the movement of the optic along the visual axis in response to natural accommodative forces of the eye. Further details regarding the accommodative mechanism 48 and the manner by which it is coupled to the optic 46 can be found in U.S. Pat. No. 7,029,497 entitled “Accommodative Intraocular Lens,” which is herein incorporated by reference.

With continued reference to FIGS. 12A and 12B, the anterior surface 46a can have a profile that can be defined as superposition of a base profile, such as the base profile defined by the above relations (2) and (3), and an auxiliary profile, such as the auxiliary profile defined by the above relations (4) and (5) or the above relation (8). A discrete phase shift across a transition region of the anterior surface can extend the depth-of-focus of the optic so as to supplement the dynamic accommodation provided by the accommodative mechanism 48.

Those having ordinary skill in the art will appreciate that various changes can be made to the above embodiments without departing from the scope of the invention. For example, one or more surface of the lenses can include a flat, rather than a curved, base profile.

What is claimed is:

1. An ophthalmic lens, comprising
at least two optics disposed in tandem along an optical axis,
an accommodative mechanism coupled to at least one of said optics and adapted to adjust a combined optical power of said optics in response to accommodative forces of an eye in which the optics are implanted so as to provide accommodation,
at least one of said optics having a surface characterized by a first refractive region, a second refractive region and a transition region therebetween,
wherein an optical phase shift across said transition region corresponds to a non-integer fraction of a design wavelength.

2. The ophthalmic lens of claim 1, wherein said accommodative mechanism is adapted to move at least one of said optics along said optical axis in response to the eye's accommodative forces so as to provide accommodation.

3. The ophthalmic lens of claim 1, wherein one of said optics provides a positive optical power and the other provides a negative optical power.

4. The ophthalmic lens of claim 3, wherein said positive optical power is in a range of about +20 D to about +60 D and said negative optical power is in a range of about −26 D to about −2 D.

5. The ophthalmic lens of claim 1, wherein at least one of said optics comprises a toric surface.

6. The ophthalmic lens of claim 1, wherein said surface having the transition region has a profile (Z_{sag}) defined by the following relation:
Z_{sag}=Z_{base}+Zₐᵤₓ,
wherein,
Z_{sag} denotes a sag of the surface relative to the optical axis as a function of radial distance from said axis and Z_{base} denotes a base profile of the surface, and wherein,
$${Z}_{ips} = \{ \begin{matrix}0 , & 0 \leq r < {r}_{1} \\ \frac{Δ}{( {r}_{1} - {r}_{1 } )} ( r - {r}_{1} ) , & {r}_{1} \leq r < {r}_{2} \\ Δ , & {r}_{2} < r\end{matrix}$$
wherein,
r₁ denotes an inner radial boundary of the transition region,
r₂ denotes an outer radial boundary of the transition region, and wherein,
Δ is defined by the following relation:
$$Δ = \frac{α λ}{( {n}_{2} - {n}_{1} )} ,$$
wherein,
n₁ denotes an index of refraction of material forming the optic,
n₂ denotes an index of refraction of a medium surrounding the optic,
λ denotes a design wavelength, and
α denotes a non-integer fraction.

7. The ophthalmic lens of claim 6, wherein
$${Z}_{base } = \frac{{cr}^{2}}{1 + \sqrt{1 - ( 1 + k ) {c}^{2} {r}^{2}}} + {a}_{2} {r}^{2} + {a}_{4} {r}^{4} + {a}_{6} {r}^{6} + … ,$$
wherein,
r denotes a radial distance from the optical axis,
c denotes a base curvature of the surface,
k denotes a conic constant,
a₂ is a second order deformation constant,
a₄ is a fourth order deformation constant, and
a₆ is a sixth order deformation constant.

8. The ophthalmic lens of claim 7, wherein said base curvature c is in a range of about 0.0152 mm⁻¹ to about 0.0659 mm⁻¹, said conic constant k is in a range of about −1162 to about −19, a₂ is in a range of about −0.00032 mm⁻¹ to about 0.0 mm⁻¹, a₄ is in a range of about 0.0 mm⁻³ to about −0.000053 (minus 5.3×10⁻⁵) mm⁻³, and a₆ is in a range of about 0.0 mm⁻⁵ to about 0.000153 (1.53×10⁻⁴) mm⁻⁵.

9. The ophthalmic lens of claim 1, wherein said surface having the transition region has a surface profile (Z_{sag}) defined by the following relation:
Z_{sag}=Z_{base}+Zₐᵤₓ,
wherein,
Z_{sag} denotes a sag of the surface relative to the optical axis as a function of radial distance from said axis, and
wherein,
$${Z}_{base} = \frac{{cr}^{2}}{1 + \sqrt{1 - ( 1 + k ) {c}^{2} {r}^{2}}} + {a}_{2} {r}^{2} + {a}_{4} {r}^{4} + {a}_{6 } {r}^{6} + … ,$$
wherein,
r denotes a radial distance from the optical axis,
c denotes a base curvature of the surface,
k denotes a conic constant,
a₂ is a second order deformation constant,
a₄ is a fourth order deformation constant, and
a₆ is a sixth order deformation constant, and
wherein,
$${z}_{aux} = \{ \begin{matrix}0 , & 0 \leq r < {r}_{1 a} \\ \frac{{Δ}_{1}}{( {r}_{1 b} - {r}_{1 a} )} ( r - {r}_{1 a} ) , & {r}_{1 a} \leq r < {r}_{1 b} \\ {Δ}_{1} , & {r}_{1 b} \leq r < {r}_{2 a} \\ {Δ}_{1} + \frac{( {Δ}_{2} - {Δ}_{1} )}{( {r}_{2 b } - {r}_{2 a} )} ( r - {r}_{2 a} ) , & {r}_{2 a} \leq r < {r}_{2 b} \\ {Δ}_{2} & {r}_{2 b} < r\end{matrix} \qquad Eq . ( X )$$
wherein
r denotes the radial distance from an optical axis of the lens,
r₁ₐ denotes the inner radius of a first substantially linear portion of transition region of the auxiliary profile,
r_{1b} denotes the outer radius of the first linear portion,
r₂ₐ denotes the inner radius of a second substantially linear portion of the transition region of the auxiliary profile, and
r_{2b} denotes the outer radius of the second linear portion, and
wherein
each of Δ₁ and Δ₂ can is defined in accordance with the following relation:
$${Δ}_{1} = \frac{{α}_{1} λ}{( {n}_{2} - {n}_{1} )} , \\ {Δ}_{2} = \frac{{α}_{2} λ}{( {n}_{2} - {n}_{1} )}$$
wherein,
n1 denotes an index of refraction of material forming the optic,
n2 denotes an index of refraction of a medium surrounding the optic,
λ denotes a design wavelength,
α₁ denotes a non-integer fraction, and
α₂ denotes a non-integer fraction.

10. The ophthalmic lens of claim 1, wherein said accommodative mechanism comprises
a ring for positioning in the capsular bag, and
a plurality of flexible members coupling the ring to at least one of said optics,
wherein said ring is adapted to cause the flexible members to move said at least one optic along the optical axis in response to accommodative forces exerted by the capsular bag to the ring.

11. The lens of claim 1, wherein said accommodative mechanism is adapted to provide a dynamic accommodation in a range of about 0.5 D to about 2.5 D.

12. The lens of claim 11, wherein said transition region is adapted to extend a depth-of-focus of said lens by at least about 0.5 D.

13. An intraocular lens system, comprising
an optical system adapted for positioning in the capsular bag of a patient's eye, said optical system comprising a plurality of lenses,
an accommodative mechanism coupled to said optical system to cause a change in an optical power of said optical system in response to natural accommodative forces of the eye so as to provide accommodation,
said optical system having at least one toric surface and at least one surface having a first refractive region, a second refractive region and a transition region therebetween,
wherein said transition region is configured such that an optical phase shift of incident light across said transition region corresponds to a non-integer fraction of a design wavelength.

14. The intraocular lens system of claim 13, wherein said design wavelength is about 550 nm.

15. The intraocular lens system of claim 13, wherein at least one of said lenses provides a positive optical power and at least another one of said lenses provides a negative optical power.

16. The intraocular lens system of claim 13, wherein said accommodative mechanism is adapted to provide dynamic accommodation in a range of about 0.5 D to about 2.5 D.

17. The intraocular lens system of claim 16, wherein said transition region extends depth-of-field of said lens system by a value in a range of about 0.5 D to about 1.25 D for pupil sizes in a range of about 2.5 mm to about 3.5 mm.

18. The intraocular lens system of claim 13, wherein said accommodative mechanism causes a relative axial movement of two of the lenses of said optical system so as to provide accommodation.

19. An intraocular lens, comprising
an optic having an anterior surface and a posterior surface,
an accommodative mechanism coupled to said optic to cause movement of said optical along visual axis in response to natural accommodative forces of an eye in which the lens is implanted so as to provide accommodation,
wherein at least one of said surfaces includes a first refractive region, a second refractive region and a transition region therebetween,
wherein an optical phase shift of incident light having a design wavelength across said transition region corresponds to a non-integer fraction of said design wavelength.
